(19) United States
(12) Patent Application Publication
(10) Pub. No.: US 2022/0301033 A1
(43) Pub. Date: Sep. 22, 2022

(54) MENU DISPLAY ANNOTATED WITH CUSTOM REQUIREMENTS

(71) Applicant: INTERNATIONAL BUSINESS MACHINES CORPORATION, Armonk, NY (US)

(72) Inventors: Ashley Kristin Silva, Durham, NC (US); Randy A. Rendahl, Raleigh, NC (US); JOCELYN SESE, Knightdale, NC (US); Frank Isaacs, Wake Forest, NC (US)

(21) Appl. No.: 17/202,456

(22) Filed: Mar. 16, 2021

(51) Int. Cl. G06Q 30/06; G06K 9/00; G06F 3/14; G16H 10/65; G16H 20/60; G06Q 50/12

(52) U.S. Cl. CPC G06Q 30/0631; G06K 9/00456; G06F 3/14; G16H 10/65; G16H 20/60; G06Q 50/12; G06K 2209/01

(57) ABSTRACT

A computer-implemented method, a computer system and a computer program product display a custom food establishment menu. The method includes receiving a plurality of ingredients for each of a plurality of food items which are displayed on a menu. The method also includes receiving a user profile that includes food ingredient requirements of a user. In addition, the method includes receiving an image of a menu and determining a name for each food item displayed on the menu using text recognition. The method further includes classifying the displayed food items based on the determined names and received user profile. Finally, the method includes displaying an image of the menu on a device which includes a visual indication associated with the classification of the displayed food items.

### BACKGROUND

Embodiments relate generally to food safety and more particularly to providing detailed information about food ingredients to people with food allergies, food intolerances, and dietary restrictions by annotating an image of a menu based on ingredient information gathered from a food establishment and food requirements gathered from a user.

Dining outside the home is a common experience in everyday life, as is the presence of smartphones and other mobile devices. Many food establishments provide their menus publicly, posting them in the window for potential diners to see and many menus are now provided electronically, e.g., via websites or other digital means, so information is widely available about the food items that are being offered in various establishments. The ubiquity of mobile devices puts this information at the fingertips of potential diners in real time, allowing for customization of menus to specific consumers.

### SUMMARY

An embodiment is directed to a computer-implemented method for displaying a custom food establishment menu. The method may include receiving a plurality of ingredients for each of a plurality of food items which are displayed on a menu. The method may also include receiving a user profile that includes food ingredient requirements of a user. In addition, the method may include receiving an image of a menu and determining a name for each food item displayed on the menu using text recognition. The method may further include classifying the displayed food items based on the determined names and received user profile. Finally, the method may include displaying an image of the menu on a device, wherein the displayed image includes a visual indication associated with the classification of the displayed food items.

In an embodiment, the classifying the displayed food items based on the determined names and the received user profile may include mapping the named food items to the received plurality of ingredients and comparing the mapped ingredients for each named food item to the food ingredient requirements from the user profile.

In other embodiments, the receiving an image of a menu may include capturing an image of the menu using a camera within the device or acquiring an image of the menu from a third-party computer server.

In another embodiment, the visual indication associated with preferred food items may include a first visual indication associated with a first type of food ingredient requirement of the user and a second visual indication associated with a second type of food ingredient requirement of the user.

In a further embodiment, the receiving a user profile may include reading information from an identification tag associated with the user.

In another embodiment, the displaying an image of the menu on a device may include displaying only food items with a specific classification.

In addition to a computer-implemented method, additional embodiments are directed to a system and a computer program product for displaying a custom food establishment menu.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### BRIEF DESCRIPTION OF THE DRAWINGS

FIG. 1 is a block diagram of an example computer system in which various embodiments may be implemented.

FIG. 2 shows an example menu being displayed by a mobile device according to an embodiment.

FIG. 3 is a flow chart diagram of a process to display a custom menu in accordance with one or more embodiments.

FIG. 4 depicts a cloud computing environment according to an embodiment.

FIG. 5 depicts abstraction model layers according to an embodiment.

### DETAILED DESCRIPTION

Eating away from home can be difficult for people with special dietary requirements. People with food allergies, sensitivities, or dietary restrictions are safest if they carefully plan their dining excursions, choosing an establishment they know is accommodating and has low or no risk. They may also do research well in advance to find and confirm that an establishment will meet their dietary needs. Being uneasy or unable to go out with friends or families on an impromptu trip to dine out could be socially awkward, isolating, stressful, and even dangerous. Using common menu information that is generally publicly available along with a smartphone or other mobile device, e.g., wearable technology such as a smart watch or smart glasses, it is possible for a user to filter the choices on an establishment's menu down to those food items that are safe or preferable and present those filtered choices in a simple and understandable way. At the same time, if a customer inputs their specific restrictions into a personal profile, this information would allow a food establishment to know that a given customer has specific needs that they can accommodate. This is also important as it has been noted that restaurants and other food establishments are often not properly notified of a food allergy by the customer with the allergy.

Food establishments commonly provide their menu publicly and many provide the menu electronically, e.g., via websites or other digital means. However, menus do not universally specify the allergens contained in each dish. Thus, many people with allergies do not have a way of knowing which dishes contain their allergens when looking at a menu, so they have a difficult time ordering food and need to ask the staff about which dishes contain specific allergens. Further, the menus that do provide information on allergens require the diner to notice the allergen information and comb through the entire menu looking for menu choices that do not contain the particular allergens of concern, which can be missed unless the diner is being very attentive.

In addition, it is common for people to have unusual allergies, e.g., an allergy to quinoa, or multiple allergies such as dairy, eggs and soy, so the question may not be as simple as asking which dishes are nut-free or dairy-free, for example. With the wide variety of allergens, it would be difficult for an establishment's staff to know which allergens, not to mention all possible combinations, are present in every menu item. As a result, after being informed of a person's restrictions, it is likely that staff then needs to check or confirm with the kitchen about a specific dish. Once they return with an answer, the customer feels like they have to make an immediate and potentially uninformed decision, so that the order for the rest of their table is not delayed. There may be follow-up questions from the customer, causing frustration and inefficiency on the part of the staff, and the whole process can be uncomfortable for the customer.

What is needed is a way to present customers with allergen information about the menu options automatically at the time of viewing the menu. This would give customers with allergies time to think about and discuss what they want to order when everyone else at their table is doing the same, rather than trying to figure out which menu choices might contain their allergens. It would also avoid the back and forth with the staff, with the added benefit of alerting the establishment that they are serving food to someone with allergies, so there should be extra care in preparing the food. This would make going out to eat a relaxing and fun experience and allow people with allergies to have the same positive experience as people without allergies. Someone with special dietary needs could pop in, unplanned, to a food establishment, open an app on a smart device, point the device's camera at the menu, and immediately see, with clear, obvious, informative, and reliable overlays, which items they can eat and which they should avoid, all according to their own self-reported preferences and needs.

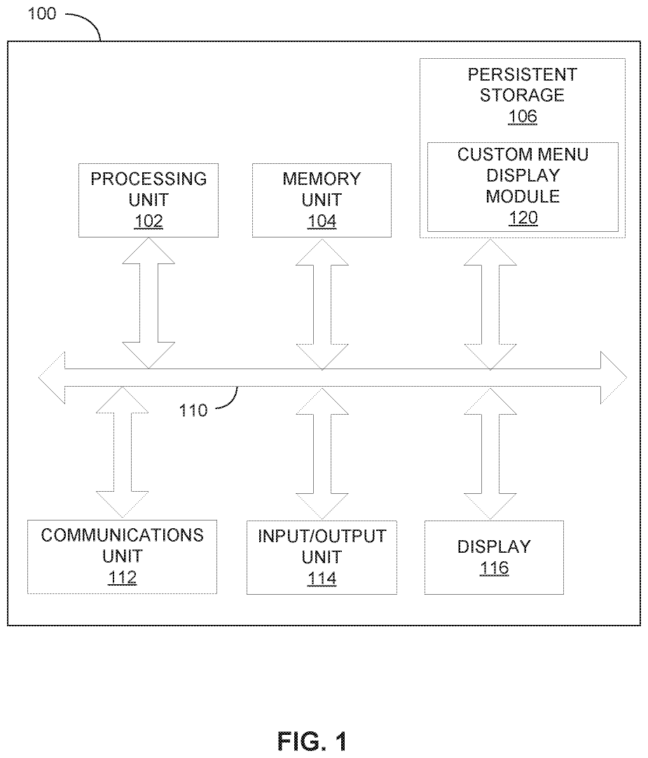

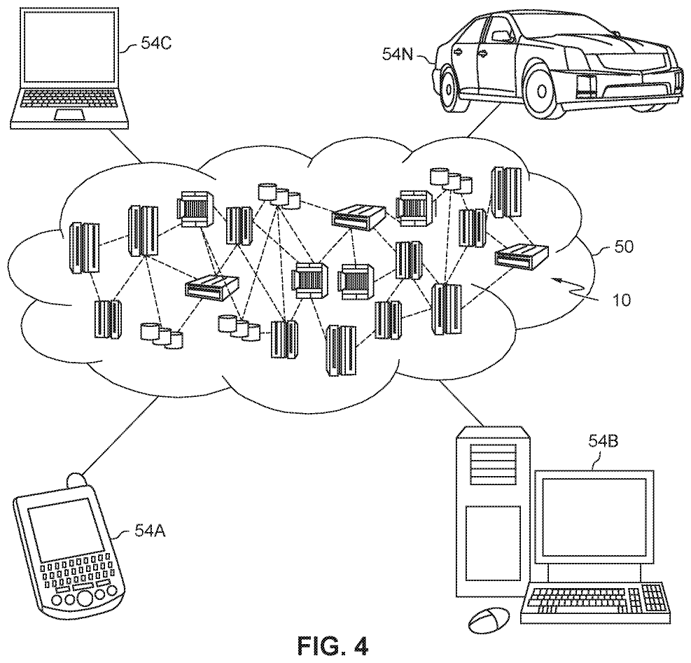

Referring now to FIG. 1, there is shown a block diagram illustrating a computer system 100 which may be embedded a cloud computing node 10 (shown in FIG. 4) in an embodiment. In another embodiment, the computer system 100 may be embedded in a “smart phone” capable of being connected to a network and which is equipped with a camera and a capability for determining location, e.g., a GPS transceiver. As shown, a computer system 100 includes a processor unit 102, a memory unit 104, a persistent storage 106, a communications unit 112, an input/output unit 114, a display 116, and a system bus 110. Computer programs such as the custom menu display module 120 may be stored in the persistent storage 106 until they are needed for execution, at which time the programs are brought into the memory unit 104 so that they can be directly accessed by the processor unit 102. The processor unit 102 selects a part of memory unit 104 to read and/or write by using an address that the processor 102 gives to memory 104 along with a request to read and/or write. Usually, the reading and interpretation of an encoded instruction at an address causes the processor 102 to fetch a subsequent instruction, either at a subsequent address or some other address. The processor unit 102, memory unit 104, persistent storage 106, communications unit 112, input/output unit 114, and display 116 interface with each other through the system bus 110.

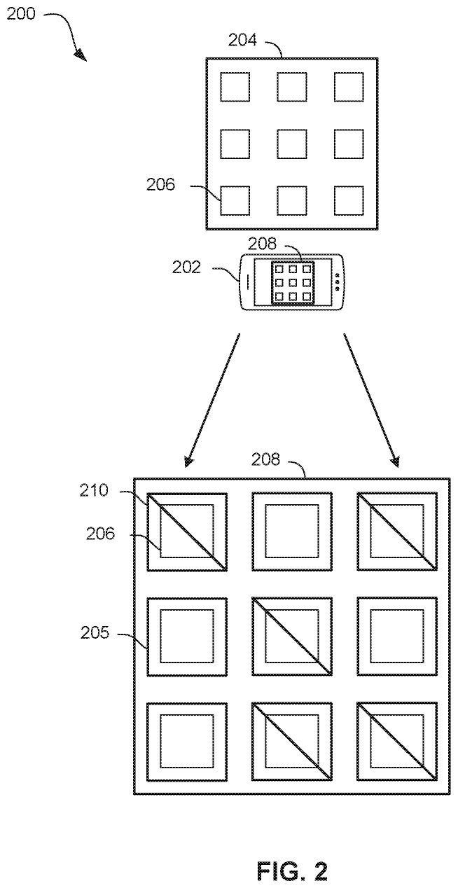

Referring to FIG. 2, an example 200 is shown of a menu 204 being displayed by a mobile device 202 according to an embodiment. The menu 204 consists of food items 206 and may be, in various embodiments, captured as an image 208 on the mobile device 202. The resulting image or custom menu 208 may be displayed on the mobile device 202 or other computer system with visual indications 210 associated with the food items 206 to indicate food items that do not meet the user's requirements. The mobile device 202 is shown in FIG. 2 as a smartphone but it should be noted that the mobile device may be any appropriate device with a screen, e.g., a laptop or tablet computing device, or wearable computing devices such as smart watches or smart glasses, to display the custom menu 208. In an embodiment, the custom menu display module 120 may place a box 205 around all the food items that are in the image and eliminate those items that do not meet the requirements by using a line through the box. The custom menu display module 120 may also use different colors for the visual indications 210, e.g., green to indicate that a food item meets the user's requirements and red to indicate that the food item does not meet the user's requirements. It should be noted that this is only one possible set of visual indications 210 and one of skill in the art would recognize that there are many possibilities for indicating that a food item does or does not meet the user's requirements, including blurring out or digitally removing those items that do not meet the requirements such that the user can only view those food items that meet their specific requirements. In addition, the custom menu 208 may include text as a visual indication 210 to more clearly indicate to the user why it has made a certain decision. This text may include the specific ingredients that has caused the item to be classified in a certain way or any other information to support a graphical visual indication 210.

In another embodiment, the user may have an image of the menu 204 loaded in the mobile device 202 ahead of time or download an image from a server on the Internet at the food establishment instead of capturing an image of the menu 204 as shown in FIG. 2 into the mobile device 202. In this embodiment, the mobile device 202 would add the visual indications 210 to the image in its memory and display a custom menu 208 to the user in the same way as FIG. 2.

As noted above, embodiments may employ any appropriate computing device with a screen. In some embodiments, the computing device may be provided by the restaurant or other food service provider. For example, a restaurant may provide their menu and a capability to place orders on a tablet computing device owned by the restaurant. Ingredient requirements and preferences could be obtained from a user, e.g., via an RFID tag. However, embodiments in which the computing device belongs to the user may be advantageous because processing is done on the customer side. The food service provider need not invest in tablet computing devices. The food service provider would only need to publish the public data feed described below. In addition, performing processing on the customer side allows the user to determine which food items meet their requirements remotely, as well as on the premises of the food service provider. It should also be noted that user privacy is more easily maintained in this model since the user profile is not published but rather used in combination with the harvested public data feed from which ingredient information is extracted.

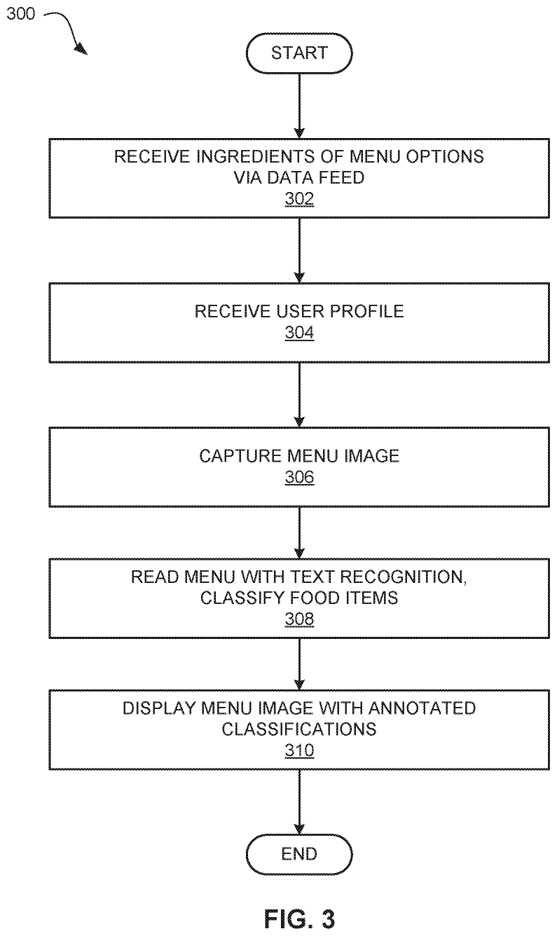

Referring to FIG. 3, an operational flowchart illustrating a process 300 for displaying a custom food establishment menu is depicted according to at least one embodiment. At 302, information may be received about the ingredients of the one or more food items on a menu. This may be done by the food establishment importing ingredient data for all food items on its menu to a public data feed that is available for download by an instance of computer system 100, e.g., a mobile device 202. The custom menu display module 120 requires a data source that contains accurate and current ingredient information for the selections on the food establishment menu. While any mechanism for the public data feed needs the ability for the food establishment to provide real-time updates, one of skill in the art will recognize that the public data feed can be provided in multiple ways. For example, the public data feed may be a locally, i.e., by the food establishment itself, hosted and maintained broadcast or may be maintained off-premises from a remote server as a third-party service. The ingredient information in the data feed may be extracted and used to map the names of the food items on the menu to the food item's ingredients.

At 304, a user profile that details the requirements for a specific user is received. A user may enter his or her ingredient requirements into a stored profile that resides on an instance of computer system 100, e.g., a personal mobile device 208 or on a central server or database that is connected to the user and retrieved by the mobile device 208 at the time of entering a food establishment. In an alternative, the ingredient requirements may be associated with an RFID tag that is worn by the user and scanned by a device in the food establishment. The user may designate an ingredient requirement as a firm requirement or a preference. A user would specify firm ingredient requirement if consumption of the ingredient causes an allergic reaction for the user. A preferred ingredient may be an ingredient a user likes or an ingredient a user prefers to avoid. It should be noted that this list is not exhaustive for how the user profile is entered into the custom menu display module 120 or provided to the food establishment. One of skill in the art would recognize that in addition to an RFID tag worn by a user, other identification technologies include near-field communication (NFC) with a mobile device 208, certain apps on a mobile device 208 or other device carried by the user, QR codes, location services on mobile devices, or any other opt-in identifying method. It is only necessary at this stage to identify the user and the specific food requirements for the user. This profile may be used as input to the custom menu display module 120 for classification of food items according to the food requirements of the user. It should be noted that the requirements for the user may be listed in any appropriate way, e.g, a list of ingredients that the user does not allow or a list that the user does allow.

At 306, an image of the menu 204 is received. As mentioned with respect to FIG. 2, this may primarily use a camera on the mobile device 208 to capture an image of the menu in the food establishment. However, it should be recognized that a user may also retrieve a stored image of the menu at any time prior to ordering, e.g., before arriving at the food establishment or while present at the establishment. It is only necessary for the custom menu display module 120 to have an image of the menu that it may use to apply visual indications 210 and display to the user. An image of the menu may be fetched, for example, from any website on a server.

At 308, the custom menu display module 120 reads the food items from the menu using known text recognition techniques such as optical character recognition (OCR) on the image. The OCR process may use a lexicon of food item names in order to improve word recognition accuracy. This yields a name corresponding to each food item on the menu and the custom menu display module 120 then classifies the food items on the menu according to the specific ingredient requirements of the user by looking up the names in the data that was received in 302 and mapping the name of each food item to its respective ingredients. These ingredients may then be compared to the ingredient requirements received in the user profile of 304. If any ingredient in the list for a given food item is identified in the requirements as not allowed for the user, the custom menu display module 120 may classify the food item as not allowed. If an ingredient is identified as a preferred ingredient, it may be classified accordingly. It should be noted that this classification process will be determined by the way that the requirements are listed in the profile. One of skill in the art would recognize that there are many possibilities for how the requirements may be listed and how the custom menu display module may interpret those requirements. It is only necessary that these techniques are aligned such that the custom menu display module may read the input data and be able to determine the specific requirements of the user.

At 310, the mobile device may render and display a digital image of the menu with visual indications that note food items that do or do not meet the requirements of the user. As noted above with respect to FIG. 2, the custom menu display module 120 may use any sort visual indication to distinguish between a food item that meets the requirements and one that does not. In addition, there may be separate visual indications that indicate degrees of requirements. For example, there may be one visual indication to indicate an allergy and another to indicate a simple user preference in addition to an indication that a food item is allowed. These indications may be overlaid on the menu such that the customer is looking at the menu as designed by the food establishment with the indications projected over the image. In another embodiment, the menu may be displayed with the non-preferred food items removed from the image such that only preferred items are displayed in the custom menu image. However, the user may not be looking at an exact representation of the physical menu of the food establishment. The menu may be shown to the user in text form with markers for the various classifications of food items. It is only necessary that the user has the information about which food items on the menu meet their requirements and those which do not so that they can make an informed ordering decision.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

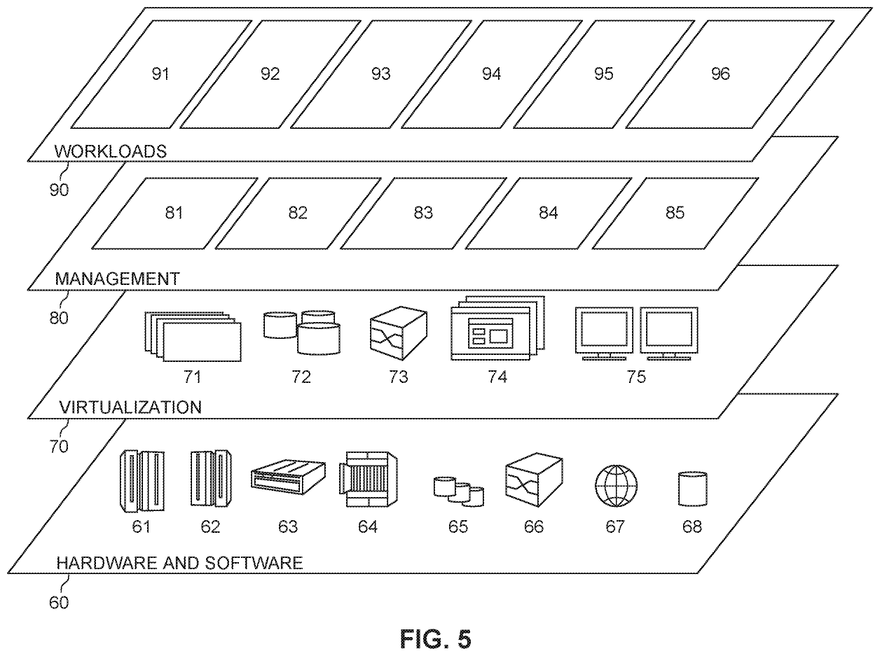

Referring now to FIG. 4, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 5 are intended to be illustrative only and that computing nodes 10 and cloud computing environment 50 can communicate with any type of computerized device over any type of network and/or network addressable connection (e.g., using a web browser).

Referring now to FIG. 5, a set of functional abstraction layers provided by cloud computing environment 50 (FIG. 4) is shown. It should be understood in advance that the components, layers, and functions shown in FIG. 5 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66, such as a load balancer. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include mapping and navigation 91; software development and lifecycle management 92; virtual classroom education delivery 93; data analytics processing 94; transaction processing 95; and other applications 96 such as the custom menu display module 120.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A computer-implemented method for displaying a custom food establishment menu, comprising:
receiving a plurality of ingredients for each of a plurality of food items, wherein the food items are displayed on a menu;
receiving a user profile, wherein the user profile includes food ingredient requirements of a user;
receiving an image of a menu;
determining a name for each food item displayed on the menu using text recognition;
classifying the displayed food items based on the determined names and received user profile; and
displaying an image of the menu on a device, wherein the displayed image includes a visual indication associated with the classification of the displayed food items.

2. The computer-implemented method of claim 1, wherein the classifying the displayed food items based on the determined names and the received user profile comprises:
mapping the named food items to the received plurality of ingredients; and
for each named food item, comparing the mapped ingredients to the food ingredient requirements from the user profile;

3. The computer-implemented method of claim 1, wherein receiving an image of a menu comprises capturing an image of the menu using a camera within the device.

4. The computer-implemented method of claim 1, wherein receiving an image of a menu comprises acquiring an image of the menu from a third-party computer server.

5. The computer-implemented method of claim 1, wherein the visual indication associated with preferred food items comprises a first visual indication associated with a first type of food ingredient requirement of the user and a second visual indication associated with a second type of food ingredient requirement of the user.

6. The computer-implemented method of claim 1, wherein receiving a user profile includes reading information from an identification tag associated with the user.

7. The computer-implemented method of claim 1, wherein the displaying an image of the menu on a device comprises displaying only food items with a specific classification.

8. A computer program product for displaying a custom food establishment menu, the computer program product comprising:
a computer readable storage device storing computer readable program code embodied therewith, the computer readable program code comprising program code executable by a computer to perform a method comprising:
receiving a plurality of ingredients for each of a plurality of food items, wherein the food items are displayed on a menu;
receiving a user profile, wherein the user profile includes food ingredient requirements of a user;
receiving an image of a menu;
determining a name for each food item displayed on the menu using text recognition;
classifying the displayed food items based on the determined names and received user profile; and
displaying an image of the menu on a device, wherein the displayed image includes a visual indication associated with the classification of the displayed food items.

9. The computer program product of claim 8, wherein the classifying the displayed food items based on the determined names and the received user profile comprises:
mapping the named food items to the received plurality of ingredients; and
for each named food item, comparing the mapped ingredients to the food ingredient requirements from the user profile;

10. The computer program product of claim 8, wherein receiving an image of a menu comprises capturing an image of the menu using a camera within the device.

11. The computer program product of claim 8, wherein receiving an image of a menu comprises acquiring an image of the menu from a third-party computer server.

12. The computer program product of claim 8, wherein the visual indication associated with preferred food items comprises a first visual indication associated with a first type of food ingredient requirement of the user and a second visual indication associated with a second type of food ingredient requirement of the user.

13. The computer program product of claim 8, wherein receiving a user profile includes reading information from an identification tag associated with the user.

14. The computer program product of claim 8, wherein the displaying an image of the menu on a device comprises displaying only food items with a specific classification.

15. A computer system for displaying a custom food establishment menu, the computer system comprising:
one or more processors, one or more computer-readable memories, one or more computer-readable tangible storage media, and program instructions stored on at least one of the one or more tangible storage media for execution by at least one of the one or more processors via at least one of the one or more memories, wherein the computer system is capable of performing a method comprising:
receiving a plurality of ingredients for each of a plurality of food items, wherein the food items are displayed on a menu;
receiving a user profile, wherein the user profile includes food ingredient requirements of a user;
receiving an image of a menu;
determining a name for each food item displayed on the menu using text recognition;
classifying the displayed food items based on the determined names and received user profile; and
displaying an image of the menu on a device, wherein the displayed image includes a visual indication associated with the classification of the displayed food items.

16. The computer system of claim 15, wherein the classifying the displayed food items based on the determined names and the received user profile comprises:
mapping the named food items to the received plurality of ingredients; and
for each named food item, comparing the mapped ingredients to the food ingredient requirements from the user profile;

17. The computer system of claim 15, wherein receiving an image of a menu comprises capturing an image of the menu using a camera within the device.

18. The computer system of claim 15, wherein receiving an image of a menu comprises acquiring an image of the menu from a third-party computer server.

19. The computer system of claim 15, wherein the visual indication associated with preferred food items comprises a first visual indication associated with a first type of food ingredient requirement of the user and a second visual indication associated with a second type of food ingredient requirement of the user.

20. The computer system of claim 15, wherein receiving a user profile includes reading information from an identification tag associated with the user.
